(19) United States
(12) Patent Application Publication
(10) Pub. No.: US 2023/0181584 A1
(43) Pub. Date: Jun. 15, 2023

(54) METHOD OF TREATING CANCER HAVING AN ACTIVATED HEDGEHOG PATHWAY

(71) Applicant: Endeavor Biomedicines, Inc., San Diego, CA (US)

(72) Inventors: Miguel de los Rios, Del Mar, CA (US); John Hood, Del Mar, CA (US)

(21) Appl. No.: 18/078,655

(22) Filed: Dec. 9, 2022

Related U.S. Application Data
(60) Provisional application No. 63/315,803, filed on Mar. 2, 2022.
(60) Provisional application No. 63/287,832, filed on Dec. 9, 2021.

(51) Int. Cl. A61K 31/506; C07K 16/28

(52) U.S. Cl. CPC A61K 31/506; C07K 16/2818

(57) ABSTRACT

The present specification provides methods for treating cancers characterized in having an activated hedgehog pathway.

### CROSS-REFERENCE TO RELATED APPLICATIONS

The present application claims the benefit of U.S. Provisional Application No. 63/287,832, filed Dec. 9, 2021, and U.S. Provisional Application No. 63/315,803, filed Mar. 2, 2022, the entire contents of each of which is incorporated by reference herein.

### FIELD

The present Specification relates to methods of cancer treatment.

### BACKGROUND

Historically, cancers have been classified and treated based on their tissue of origin. However, any particular type of cancer (as judged by tissue of origin) may arise from any of multiple distinct underlying lesions. This was not a major issue when most pharmaceuticals for the treatment of cancer were (broadly) cytotoxic agents targeting the rapid growth of tumor cells. However, as understanding of the mechanisms of tumorigenesis and neoplasia have become more sophisticated, and drugs have been developed based on those mechanisms, treatment of cancer based on its affected tissue or origin has become much less satisfactory.

Any particular mechanism may account for only a small percentage of cancers of a particular tissue of origin, but may be operable in multiple types. Without being able to apply a mechanism-targeted treatment specifically to patients in whose cancers that mechanism is operable, one is left to treat the relevant types of cancer generally and observe which patients respond. This leads to ineffective treatment and lost opportunity for the patient to receive a different treatment that may be more suitable for their disease.

One long-known oncogenic mechanism operable in a variety of cancer types is aberrant activation of the Hedgehog (Hh) signaling pathway. However, a clinically useful treatment targeting this pathway has yet to be developed. This owes in part to:
a. the unacceptable toxicity of Hh pathway inhibitors, such as vismodegib and saridegib (IPI-926); and
b. that historically, patients have been selected on the basis of cancer type (tissue of origin).

### SUMMARY

It is disclosed herein that certain 1,4-disubstituted phthalazine compounds that are potent inhibitors of Smoothened (SMO; a G protein-coupled receptor that is a component of the hedgehog signaling pathway and is conserved from flies to humans) and the downstream transcription factors Gli1 and Gli2, exhibit a desirable toxicology profile, thus addressing part of this issue.

The other part of the issue is now addressed by the recognition of various Hh pathway-activating mutations that can be used to identify tumors that are susceptible to treatment with these compounds. Thus, disclosed herein are compositions and methods for treating cancers having an activated Hh signaling pathway. One aspect is a method of treating cancer in a patient in need thereof, wherein the cancer is characterized in having an activated hedgehog pathway, comprising administering an effective amount of a compound of Formula I:

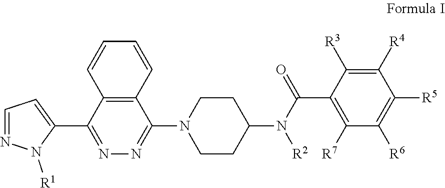

wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

One aspect is a method of treating cancer by identifying a patient with a tumor characterized by comprising means for activating a hedgehog pathway and administering an effective amount of a compound of Formula I to the patient.

One aspect is a method of treating cancer by detecting means for activating a hedgehog pathway and administering an effective amount of a compound of Formula I to the patient.

A further aspect is a method of treating cancer in a patient in need thereof, wherein the cancer is characterized in having an activated hedgehog pathway, comprising:
c. administering an effective amount of a compound of Formula I;

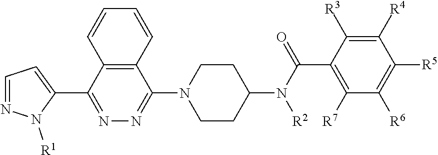

wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof; and administering an effective amount of an anti-cancer agent such as an immuno/oncology (I/O) antibody to the patient.

A further aspect is a method of treating cancer by identifying a patient with a tumor characterized by comprising means for activating a hedgehog pathway and administering an effective amount of a compound of Formula I to the patient; and administering an effective amount of an anti-cancer agent such as an I/O antibody to the patient.

A further aspect is a method of treating cancer by detecting means for activating a hedgehog pathway and administering an effective amount of a compound of Formula I to the patient; and administering an effective amount of an anti-cancer agent such as an I/O antibody to the patient.

In some embodiments, the compound of Formula I is 4-Fluoro-N-methyl-N-(1-(4-(1-methyl-1H-pyrazol-5-yl)phthalazin-1-yl)piperidin-4-yl)-2-(trifluoromethyl)benzamide (CAS 1258861-20-9):

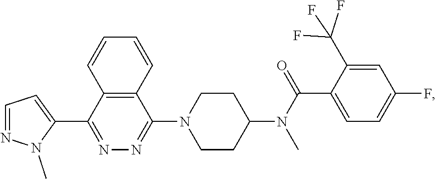 [SMILES: CN(C(=O)c1ccc(F)cc1C(F)(F)F)C1CCN(c2nnc(-c3ccnn3C)c3ccccc23)CC1]

also known as taladegib, or a pharmaceutically acceptable salt thereof.

In some embodiments, the compound of Formula I, for example, taladegib, or a pharmaceutically acceptable salt thereof, is provided in a pharmaceutical composition further comprising a pharmaceutically acceptable excipient, carrier or diluent.

In some embodiments, the administration of the compound of Formula I, for example, taladegib, or a pharmaceutically acceptable salt thereof, is combined with administration of at least two anti-cancer agents.

Further embodiments comprise selection of patients based upon a physiological characteristic, such as, for example, PD-L1 expression.

### BRIEF DESCRIPTION OF THE DRAWINGS

FIG. 1A-C depicts the topology and structure of PTCH1. FIG. 1A presents the amino acid coordinates of the topological regions of the protein. FIG. 1B presents a ribbon diagram of the protein structure. FIG. 1C lists missense, insertion, or deletion mutations in PTCH1 that may result in loss of function.

FIG. 2 depicts the location and frequency of mutations in PTCH1, including the most frequent single-base insertions/deletions, resulting in truncated protein.

FIG. 3 depicts the topology of SMO by presenting the amino acid coordinates of the topological regions of the protein.

FIG. 4 depicts the location and frequency of mutations in SMO, including the most frequent insertions/deletions and missense amino acid changes.

FIG. 5 depicts the general topology and location of most frequent mutations in SMO.

### DESCRIPTION

Taladegib is a known inhibitor of smoothened (SMO); a regulator of the hedgehog signaling pathway. Taladegib has been studied in phase I clinical trials in patients chosen on the basis of cancer tissue of origin, for example, basal cell carcinoma (BCC) and small cell lung cancer, or stage of disease, for example advanced solid tumors. One study of esophageal cancer additionally required nuclear Gli1 labeling. An alternative approach to the development and use of cancer treatments is based upon the underlying mechanism of individual cancers, rather than on their classification as to tissue or origin and stage of disease. However, to utilize this approach one must first be able to identify the operative underlying mechanism in an individual cancer and also have a tolerable drug that disrupts that mechanism.

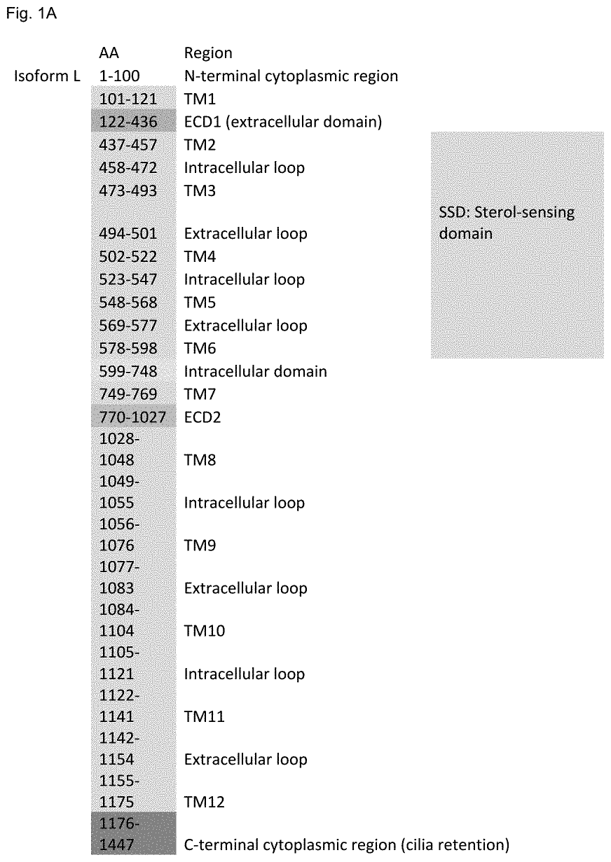

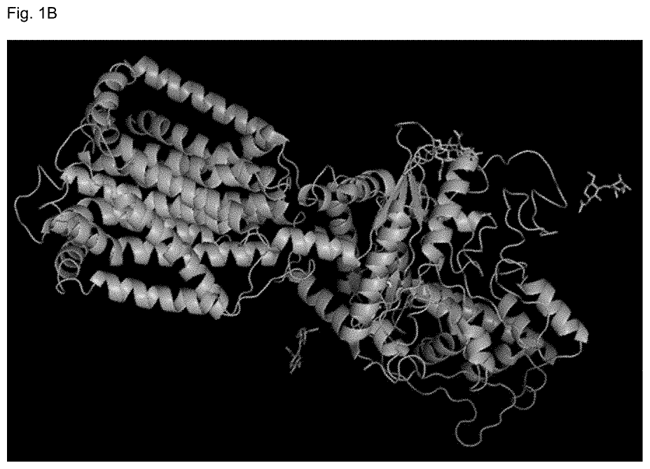

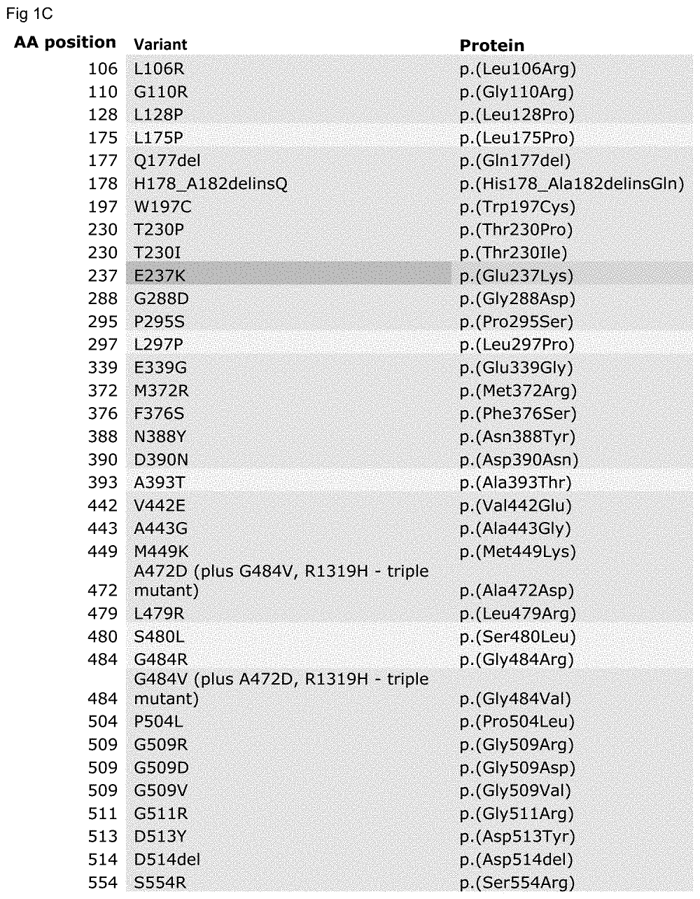

Disclosed cancer treatments adopt this strategy, seeking to apply SMO inhibitors, of which taladegib is a prime example, to the treatment of cancers characterized by an activated hedgehog signaling pathway and where the activating mechanism can be inhibited by a SMO inhibitor. Gli1 can be activated downstream of SMO, so that a SMO inhibitor will not necessarily inhibit tumor characterized by Gli1 activation. Mutations that can lead to an activation of the hedgehog signaling pathway and that can be reversed by SMO inhibition include patched1 (Ptch1) loss-of-function mutations, SMO gain-of-function mutations, and sonic hedgehog (SHH) gain-of-function mutations. Mutations activating the hedgehog pathway are found in >5% of all cancers. In some embodiments, compounds of Formula I are referred to as means for inhibiting SMO or means for inhibiting Gli1 activity. In some embodiments, these means specifically exclude compounds having one or more of the alternative substituents for the variable positions of Formula I. In one aspect, the hedgehog pathway is activated by a loss-of-function mutation in PTCH1, so that PTCH1 fails to inhibit SMO even in the absence of a hedgehog ligand such as sonic hedgehog (SHH). In some embodiments, the loss-of-function mutation in PTCH1 is a frameshift mutation resulting in a truncated protein. In some embodiments the truncating frameshift mutation comprises N97Tfs*20, N97Kfs*43, S1203Afs*52, R1308Efs*64, or R1308Qfs*17. In some embodiments, the loss-of-function mutation in PTCH1 is a missense, insertion, or deletion mutation, for example, a mutation listed in FIG. 1C. In some embodiments, the cancer comprises endometrial cancer, colorectal cancer, pineal cancer, head and neck cancer, bladder cancer, glioma, Wilms cancer, bone cancer, cervical cancer, mesothelioma, sarcoma, ovarian cancer, pancreatic cancer, renal cell carcinoma, non-small cell lung cancer, small cell lung cancer, breast cancer, esophageal cancer, prostate cancer, basal cell carcinoma, or medulloblastoma. The PTCH1 loss-of-function mutations can be observed in other cancers, at reduced frequencies, but can be treated according to the disclosed methods as well.

In one aspect, the hedgehog pathway is activated by a gain-of-function mutation in SMO, so that SMO is active even in the presence of PTCH1. In some embodiments, the gain-of-function mutation is an insertion or deletion in the signal sequence. In some embodiments, the insertion or deletion comprises L23dup, L23del, or L22L23dup. In some embodiments, the gain-of-function mutation is a missense mutation in the N-terminal extracellular domain. In some embodiments, the missense mutation is E194K. In some embodiments, the gain-of-function mutation is a missense mutation in the first transmembrane domain. In some embodiments, the missense mutation in the first transmembrane domain is A235V. In some embodiments, the gain-of-function mutation is a missense mutation in the fifth transmembrane domain. In some embodiments, the missense mutation in the fifth transmembrane domain is L412F. In some embodiments, the gain-of-function mutation is a missense mutation in the seventh transmembrane domain. In some embodiments, the missense mutation in the seventh transmembrane domain is W535L. In some embodiments, the gain-of-function mutation is a frameshift mutation in the C-terminal intracellular domain. In some embodiments, the frameshift mutation in the C-terminal intracellular domain is P694Lfs*. In some embodiments, the gain-of-function mutation is a missense mutation in the C-terminal intracellular domain. In some embodiments, the missense mutation in the C-terminal intracellular domain is R562Q. In some embodiments, the missense mutation is P641A.

In some embodiments, the cancer comprises meningioma, medulloblastoma, basal cell carcinoma, colorectal cancer, pineal cancer, head and neck cancer, bladder cancer, glioma, non-small cell lung cancer, Wilms cancer, small cell lung cancer, bone cancer, cervical cancer, mesothelioma, sarcoma, prostate cancer, ovarian cancer, breast cancer, pancreatic cancer, renal cell carcinoma, or endometrial cancer. The SMO gain-of-function mutations can be observed in other cancers, at reduced frequencies, but can be treated according to the disclosed methods as well.

In one aspect the hedgehog pathway is activated by a gain-of-function mutation in SHH, so that SHH binds directly to SMO and activates it even in the presence of PTCH1.

In some embodiments, the herein-disclosed mutations constitute means for activating a hedgehog pathway. Some embodiments specifically exclude one or more of these mutations or classes of mutations.

Further, in embodiments, the presence of an activated hedgehog pathway can be determined by its affect upon other biological mechanisms. For example, in cases, the activated hedgehog pathway increases PD-L1 expression. Thus, embodiments can comprise the administration of an anti PD-1 or PD-L1 antibody, for example a monoclonal anti PD-1 or PD-L1 antibody, in combination with a compound of Formula 1. Further embodiments can comprise the administration of an anti PD-1 or PD-L1 antibody, for example a monoclonal anti PD-1 or PD-L1 antibody, in combination with a compound of Formula 1, and further in combination with an additional anticancer agent.

### Definitions

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Patient” means a human or non-human subject receiving medical or veterinary care.

“Pharmaceutically acceptable carrier, diluent, or excipient” is a medium generally accepted in the art for the delivery of biologically active agents to mammals, e.g., humans. The compounds of the present disclosure can be formulated as pharmaceutical compositions or formulations using a pharmaceutically acceptable carrier, diluent, or excipient and administered by a variety of routes. In particular embodiments, such compositions are for oral or intravenous administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

“Pharmaceutically acceptable salts” refers to the relatively non-toxic, inorganic and organic salts of compounds of the present disclosure. The compounds of the present disclosure are capable of reaction, for example, with a number of inorganic and organic acids to form pharmaceutically acceptable acid addition salts. Such pharmaceutically acceptable salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol 66, No. 1, January 1977.

“Pharmaceutical composition” means a formulation comprising an active ingredient. The word “formulation” means that there is at least one additional ingredient (such as, for example and not limited to, an albumin [such as a human serum albumin or a recombinant human albumin] and/or sodium chloride) in the pharmaceutical composition. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Therapeutic formulation” means a formulation that can be used to treat and thereby alleviate a disorder or a disease and/or symptom associated thereof.

“Therapeutically effective amount” means the level, amount or concentration of an agent needed to treat a disease, disorder or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of an symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition. The term “treating” or “treatment” broadly includes any kind of treatment activity, including the diagnosis and mitigation of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. This includes, for example, direction to the patient to undergo, or to a clinical laboratory to perform, a diagnostic procedure, such as an assessment of mutations associated with activation of a hedgehog pathway as disclosed herein so that ultimately the patient may receive the benefit thereof including appropriate treatment. In some embodiments, the orders, instructions, and advice aspect of treatment activity can also include encouraging, inducing, or mandating that a particular medicament or test, or combination thereof, be chosen for treatment of a condition—and the medicament is actually used—by approving insurance coverage for the medicament or test, denying coverage for an alternative medicament or test, including the medicament or test on, or excluding an alternative medicament or test, from a drug formulary, or offering a financial incentive to use the medicament or test, as might be done by an insurance company or a pharmacy benefits management company, and the like.

In some embodiments, treatment activity can also include encouraging, inducing, or mandating that a particular medicament or test be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

Disclosed embodiments comprise methods of treating cancer in patients in need thereof, wherein the cancer is characterized in having an activated hedgehog pathway, the methods comprising administering an effective amount of a compound of Formula I:

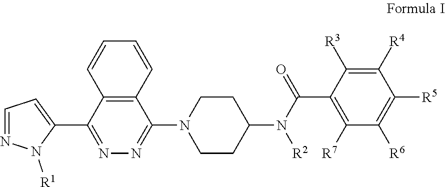

wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof. Under standard nomenclature used throughout this disclosure, the terminal portion of the designated side chain is described first, followed by the adjacent functionality toward the point of attachment. For example, a methylsulfonyl substituent is equivalent to CH₃—SO₂—. Compounds of Formula I and their synthesis are described in U.S. Pat. No. 9,000,023, which is hereby incorporated by reference in its entirety.

Further embodiments can comprise administering an effective amount of an I/O antibody. In embodiments, the I/O antibody comprises a monoclonal I/O antibody. For example, the I/O antibody can comprise a tumor-directed monoclonal antibody. When administered to a cancer-bearing patient, a tumor-directed monoclonal antibody can mark tumor cells for destruction, interfere with immune receptor signaling, promote immune receptor degradation, and deliver anti-cancer agents directly to tumor cells. In embodiments, the tumor-directed monoclonal antibody can comprise an anti PD-1 or PD-L1 antibody.

Further embodiments comprise methods of treating cancer by identifying a patient with a tumor characterized by comprising means for activating a hedgehog pathway and administering an effective amount of a compound of Formula I, or a pharmaceutically acceptable salt thereof, to the patient. Disclosed methods can further comprise administration of an effective amount of an antibody, for example an I/O antibody such as a monoclonal I/O antibody. In embodiments, the tumor-directed monoclonal antibody can comprise an anti PD-1 or PD-L1 antibody.

Further embodiments comprise methods of treating cancer by detecting means for activating a hedgehog pathway and administering an effective amount of a compound of Formula I, or a pharmaceutically acceptable salt thereof, to the patient. Disclosed methods can further comprise administration of an effective amount of an antibody, for example an I/O monoclonal antibody. In embodiments, the tumor-directed monoclonal antibody can comprise an anti PD-1 or PD-L1 antibody.

In some embodiments, compounds of Formula I are referred to as means for inhibiting SMO or means for inhibiting Gli1 activity. In some embodiments, these means specifically exclude compounds having one or more of the alternative substituents for the variable positions of Formula I. For example, in some embodiments the means exclude a compound of Formula I, wherein R¹ is hydrogen, or wherein R² is hydrogen, or wherein R³ is not trifluoromethyl, or wherein R⁵ or R⁶ or R⁷ is not hydrogen, or wherein R⁵ is not fluoro, or any combination thereof. In further embodiments, the means exclude a compound of Formula I requiring or excluding any alternative substituent of R¹ though R⁷, alone or in any combination.

With respect to the cancer being characterized in having an activated hedgehog pathway, identifying a patient with a tumor characterized by comprising means for activating a hedgehog pathway, and detecting means for activating a hedgehog pathway, it is noted that it has already become common to biopsy a patient's tumor and/or blood to determine what cancer related mutations may be present, including mutations in PTCH1, SMO and SSH. Determination of mutations in tumor samples and/or blood samples commonly employ sequencing of DNA or RNA. As used herein, cancer being characterized in having an activated hedgehog pathway refers specifically to ligand-independent activation. Over-expression of Hh ligand can also cause activation of the Hh pathway, but cancers arising from this mechanism are not an object of the disclosed methods of treatment, nor is such over-expression to be considered means for activating a hedgehog pathway as used herein.

In some embodiments, the compound of Formula I is 4-Fluoro-N-methyl-N-(1-(4-(1-methyl-1H-pyrazol-5-yl)phthalazin-1-yl)piperidin-4-yl)-2-(trifluoromethyl)benzamide (CAS 1258861-20-9):

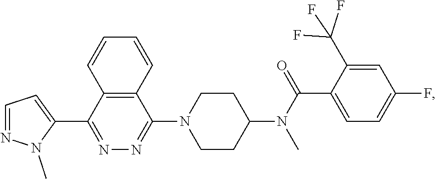 [SMILES: CN(C(=O)c1ccc(F)cc1C(F)(F)F)C1CCN(c2nnc(-c3ccnn3C)c3ccccc23)CC1]

also known as taladegib, or a pharmaceutically acceptable salt thereof.

In some embodiments, the compound of Formula I, for example, taladegib, or a pharmaceutically acceptable salt thereof, is administered orally. In some embodiments, taladegib is administered at a dosage of 50-200 mg, for example, 50, 75, 100, 125, 150, 175, or 200 mg, or in a range bound by any pair of those values. In some embodiments the dosage is administered, for example, every day, every 2 days, every 3 days, every 1 to 3 days, or the like.

In some embodiments, the compound of Formula I, for example, taladegib, or a pharmaceutically acceptable salt thereof, is provided in a pharmaceutical composition. For example, in embodiments, disclosed compositions can comprise a combination of an I/O antibody and a compound of Formula 1.

Compounds of Formula I inhibit Gli1 activity, generally with an IC₅₀ of <40 nM, as measured in Daoy cells and described in U.S. Pat. No. 9,000,023. Taladegib has an IC₅₀ of about 2.4 nM in this assay.

In some embodiments, treatment further comprises administering an additional anti-cancer agent. In some embodiments the additional anticancer agent is a traditional chemotherapeutic agent, for example, carboplatin, cisplatin, gemcitabine, etoposide, or paclitaxel. In some embodiments the additional anti-cancer agent is another targeted therapy, for example, samotolisib, crenigacestat, or abemaciclib. In some embodiments, the additional anti-cancer agent is radiation therapy. In some embodiments, the additional anticancer agent is an immune checkpoint inhibitor.

Immune checkpoint inhibition therapy refers to the use of pharmaceuticals, typically biologics, that act on regulatory pathways in the differentiation and activation of T cells to promote the passage of T cell developmental program through these checkpoints so that anti-tumor (or other therapeutic) activity can be realized. The agents bringing about immune checkpoint therapy are commonly called immune checkpoint inhibitors and it should be understood that it is the check on T cell development that is being inhibited. Thus, while many immune checkpoint inhibitors also inhibit the interaction of receptor-ligand pairs (e.g., programmed cell death 1 (PD-1) interaction with programmed death-ligand 1 (PD-L1)), other checkpoint inhibitors (such as anti-OX40, anti GITR, anti-CD137, anti-CD122, anti-CD40, and anti-ICOS) act as agonists of targets that release or otherwise inhibit the check on T cell development, ultimately promoting effector function and/or inhibiting regulatory function. While inhibition of some checkpoints has proven to be sufficient to mediate clinical improvement in some instances, inhibition of other checkpoints works best in combinations. Most commonly, antibodies against one member of the receptor-ligand pair are used. In alternative embodiments, the antibody is replaced with another protein that similarly binds to the immune checkpoint target molecule. In some instances, these non-antibody molecules comprise an extracellular portion of the immune checkpoint target molecule's ligand or binding partner, that is, at least the extracellular portion needed to mediate binding to the immune checkpoint target molecule. In some embodiments, this extracellular binding portion of the ligand is joined to additional polypeptide in a fusion protein. In some embodiments, the additional polypeptide comprises an Fc or constant region of an antibody.

Programmed death-1 (PD-1) is a checkpoint protein on T cells. Antibodies against both PD-1 and its binding partner programmed death-ligand 1 (PD-L1) have been used clinically as immune checkpoint inhibitors (PD-1 blockade). Non-limiting examples of monoclonal antibodies (mAbs) that target PD-1/PD-L1 include: the anti-PD-1 mAbs nivolumab (OPDIVO®, Bristol-Myers Squibb), pembrolizumab (KEYTRUDA®, Merck & Co.), cemiplimab-rwlc (LIBTAYO®, Regeneron Pharmaceuticals), and the anti-PD-L1 mAbs durvalumab (MED14736, IMFINZI™, Medimmune), atezolizumab (MPDL3280A; TECENTRIQ®, Hoffmann-La Roche), avelumab (BAVENCIO®, EMD Serono), and BMS-936559 (Bristol-Myers Squibb). These may be referred to as means for PD-1 blockade, means for inhibiting PD-1/PD-L1 binding, or means for immune checkpoint inhibition or immune checkpoint inhibitors. Disclosed embodiments comprise administration of the compound of Formula I, for example, taladegib, or a pharmaceutically acceptable salt thereof, in combination with administration of means for immune checkpoint inhibition. In embodiments, the means for immune checkpoint inhibition can comprise an anti PD-1 or anti PD-L1 antibody, for example a monoclonal anti PD-1 or PD-L1 antibody.

Embodiments can comprise administration of the compound of Formula I, for example, taladegib, or a pharmaceutically acceptable salt thereof, in combination with administration of means for immune checkpoint inhibition, and further in combination with administration of an additional anti-cancer agent. CTLA-4 is an immune checkpoint molecule expressed on the surface of CD4 and CD8 T cells and on CD25+, FOXP3+T regulatory (Treg) cells. Non-limiting examples of monoclonal antibodies that target CTLA-4 include ipilimumab (YERVOY®; Bristol-Myers Squibb) and tremelimumab (Medimmune). These may be referred to as means for inhibiting CTLA-4, or means for immune checkpoint inhibition or immune checkpoint inhibitors.

TIM-3 (T-cell immunoglobulin and mucin-domain containing-3) is a molecule selectively expressed on IFN-γ-producing CD4⁺ T helper 1 (Th1) and CD8⁺ T cytotoxic 1 (Tc1) T cells. Non-limiting, exemplary antibodies to TIM-3 are disclosed in U.S. Patent Application Publication 20160075783 which is incorporated by reference herein for all it contains regarding anti-TIM-3 antibodies. Other anti-TIM-3 antibodies include TSR-022 (Tesaro). These may be referred to as means for inhibiting TIM-3, or means for immune checkpoint inhibition or immune checkpoint inhibitors.

Disclosed embodiments comprise administration of the compound of Formula I, for example, taladegib, or a pharmaceutically acceptable salt thereof, in combination with administration of means for immune checkpoint inhibition. In embodiments, the means for immune checkpoint inhibition can comprise an anti CTLA-4 antibody, for example a monoclonal anti CTLA-4 antibody. LAG-3 (lymphocyte-activation gene 3; CD223) negatively regulates cellular proliferation, activation, and homeostasis of T cells, in a similar fashion to CTLA-4 and PD-1 and plays a role in Treg suppressive function. Non-limiting exemplary antibodies to LAG-3 include GSK2831781 (GlaxoSmithKline), relatlimab (BMS-986016, Bristol-Myers Squibb), and the antibodies disclosed in U.S. Patent Application Publication 2011/0150892 which is incorporated by reference herein for all it contains regarding anti-LAG-3 antibodies. These may be referred to as means for inhibiting LAG-3, or means for immune checkpoint inhibition or immune checkpoint inhibitors.

Disclosed embodiments comprise administration of the compound of Formula I, for example, taladegib, or a pharmaceutically acceptable salt thereof, in combination with administration of means for immune checkpoint inhibition. In embodiments, the means for immune checkpoint inhibition can comprise an anti LAG-3 antibody, for example a monoclonal anti LAG-3 antibody.

TIGIT (T cell immunoreceptor with Ig and ITIM domains) is an immunoreceptor inhibitory checkpoint that has been implicated in tumor immunosurveillance. It competes with immune activating receptor CD226 (DNAM-1) for the same set of ligands: CD155 (PVR or poliovirus receptor) and CD112 (Nectin-2 or PVRL2). Anti-TIGIT antibodies have demonstrated synergy with anti-PD-1/PD-L1 antibodies in pre-clinical models. Tiragolumab (Roche), etigilimab (OncoMed), vibostolimab (MK-7684; Merck), and EOS-448 (iTeos Therapeutics) are non-limiting examples of an anti-TIGIT antibodies. They may be referred to as means for inhibiting TIGIT, or means for immune checkpoint inhibition or immune checkpoint inhibitors.

Disclosed embodiments comprise administration of the compound of Formula I, for example, taladegib, or a pharmaceutically acceptable salt thereof, in combination with administration of means for immune checkpoint inhibition. In embodiments, the means for immune checkpoint inhibition can comprise an anti TIGIT antibody, for example a monoclonal anti TIGIT antibody.

GITR (glucocorticoid-induced TNFR-related protein) promotes effector T cell functions and inhibits suppression of immune responses by regulatory T cells. As with OX-40, mentioned above, the checkpoint inhibitor is an agonist of the target, in this case GITR. An agonistic antibody, TRX518 is currently undergoing human clinical trials in cancer. While by itself it may not be sufficient to mediate substantial clinical improvement in advanced cancer, combination with other checkpoint inhibition, such as PD-1 blockade was promising.

Disclosed embodiments comprise administration of the compound of Formula I, for example, taladegib, or a pharmaceutically acceptable salt thereof, in combination with administration of means for immune checkpoint inhibition. In embodiments, the means for immune checkpoint inhibition can comprise an anti GITR antibody, for example a monoclonal anti GITR antibody.

Other immune checkpoint inhibitor targets comprise B- and T-cell attenuator (BTLA), CD40, CD122, inducible T-cell costimulator (ICOS), OX40 (tumor necrosis factor receptor superfamily, member 4), Siglec-15, B7H3, CD137 (4-1BB; as with CD40 and OX40, checkpoint inhibition is accomplished with an agonist) and others are potentially useful in the disclosed methods. Several anti-OX40 agonistic monoclonal antibodies are in early phase cancer clinical trials including, but not limited to, MED10562 and MED16469 (Medimmune), MOXR0916 (Genetech), and PF-04518600 (Pfizer); as is an anti-ICOS agonistic antibody, JTX-2011 (Jounce Therapeutics). Anti-CD40 agonistic antibodies under clinical investigation include dacetuzumab, CP-870,893 (selicrelumab), and Chi Lob 7/4. Anti-siglec-15 antibodies are also known (see, for example, U.S. Pat. No. 8,575,531). Anti-CD137 agonistic antibodies include, but are not limited to, urelumab and utomilumab. Additionally, CD122 has been targeted in cancer clinical trials with bempegaldesleukin (NKTR-214, a pegyltated-IL-2 used as a CD122-biased agonist). B7H3 has been targeted both for immune checkpoint inhibition and as a tumor antigen with reagents such as enoblituzumab, ¹³¹I-omburtamab, ¹⁷⁷Lu-DTPA-omburtamab, ¹³¹I-8H9, ¹²⁴I-8H9, MCG018, and DS-7300a. These may be referred to as means for immune checkpoint inhibition or means for inhibiting (or activating (agonizing), as appropriate) their respective targets. In embodiments, the additional anticancer agent comprises an I/O antibody. In embodiments, the I/O antibody can comprise, for example, an antibody listed in Table 1:

| | | |
|---|---|---|
| Name | Antigen | Format |

Unconjugated Antibodies

| | | |
|---|---|---|
| Atezolizumab | PD-L1 | Humanized IgG1 |
| Avelumab | PD-L1 | Human IgG1 |
| Bevacizumab | VEGF | Humanized IgG1 |
| Cemiplimab | PD-1 | Human IgG4 |
| Cetuximab | EGFR | Chimeric IgG1 |
| Daratumumab | CD38 | Human IgG1 |
| Dinutuximab | GD2 | Chimeric IgG1 |
| Durvalumab | PD-L1 | Human IgG1 |
| Elotuzumab | SLAMF7 | Humanized IgG1 |
| Ipilimumab | CTLA-4 | Human IgG1 |
| Isatuximab | CD38 | Chimeric IgG1 |
| Mogamulizumab | CCR4 | Humanized IgG1 |
| Necitumumab | EGFR | Human IgG1 |
| Nivolumab | PD-1 | Human IgG4 |
| Obinutuzumab | CD20 | Humanized IgG2 |
| Ofatumumab | CD20 | Human IgG1 |
| Olaratumab | PDGFRα | Human IgG1 |
| Panitumumab | EGFR | Human IgG2 |
| Pembrolizumab | PD-1 | Humanized IgG4 |
| Pertruzumab | HER2 | Humanized IgG1 |
| Ramucirumab | VEGFR2 | Human IgG1 |
| Rituximab | CD20 | Chimeric IgG1 |
| Trastuzumab | HER2 | Humanized IgG1 |

Antibody-Drug Conjugates

| | | |
|---|---|---|
| Gemtuzumab | CD33 | Humanized ADC |
| ozogamicin | | |
| Brentuximab | CD30 | Chimeric ADC |
| vedotiv | | |
| Trastuzumab | HER2 | Humanized ADC |
| emtansine | | |
| Inotuzumab | CD22 | Humanized ADC |
| ozogamacin | | |
| Polatuzumab | CD79B | Humanized ADC |
| vedotin | | |
| Enfortumab vedotin | Nectin-4 | Human ADC |
| Trastuzumab | HER2 | Humanized ADC |
| deruxtecan | | |
| Sacituzumab | TROP2 | Humanized ADC |
| govitecan | | |
| Moxetumomab | CD22 | Mouse ADC |
| pasudotox | | |
| Ibritumomab | CD20 | Mouse IgG1-Y90 |
| tiuxetan | | or In111 |
| Iodine(I131) | CD20 | Mouse IgG2-I131 |
| tositumomab | | |
| Blinatumomab | CD19, CD3 | Mouse BiTE |

In embodiments, the I/O antibody can comprise, for example, an anti-PD-1 antibody such as nivolumab. Some cancer cells have large amounts of PD-1, which helps them “hide” from an immune attack. Monoclonal antibodies that target either PD-1 or PD-L1 can block this binding and boost the immune response against cancer cells.

In embodiments, the I/O antibody can comprise multiple antibodies.

In embodiments comprising administration of an I/O antibody, the antibody can be administered, for example, orally, or parenterally, for example intramuscularly or intravenously. In embodiments, the dosage of the I/O antibody can be based upon body weight. For example, in embodiments, the dosage of the I/O antibody can be 25 mg/Kg, 30 mg/Kg, 35 mg/Kg, 40 mg/Kg, 45 mg/Kg, 50 mg/Kg, 55 mg/Kg, 60 mg/Kg, 65 mg/Kg, 70 mg/Kg, 75 mg/Kg, 80 mg/Kg, 85 mg/Kg, 90 mg/Kg, 95 mg/Kg, 100 mg/Kg, 150 mg/Kg, 200 mg/Kg, or the like. In embodiments, the dosage of the I/O antibody can be a “fixed” amount, such as, for example, 20 mg, 30 mg, 40 mg, 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, or the like.

In embodiments, the I/O antibody is provided in a pharmaceutical composition further comprising a pharmaceutically acceptable excipient, carrier or diluent.

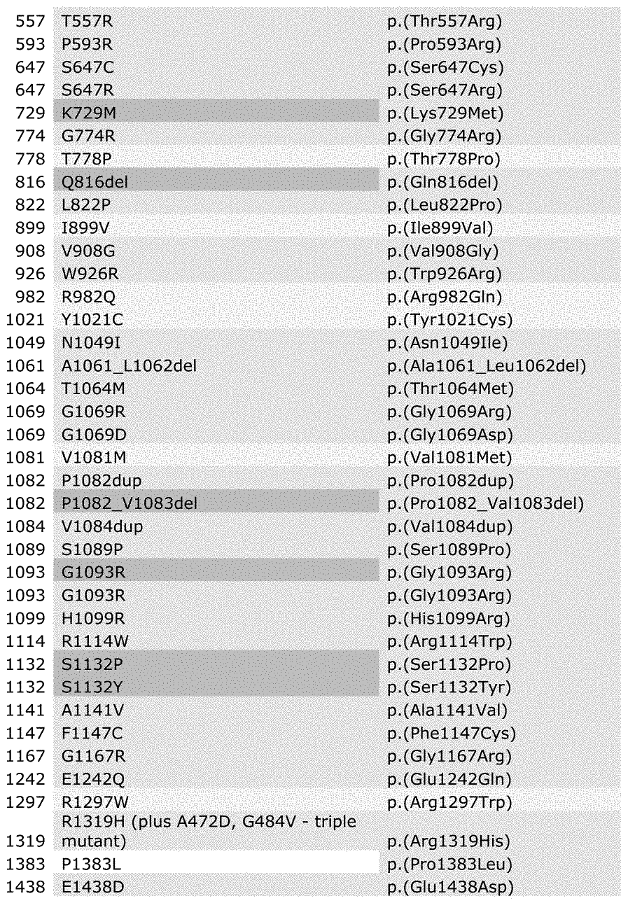

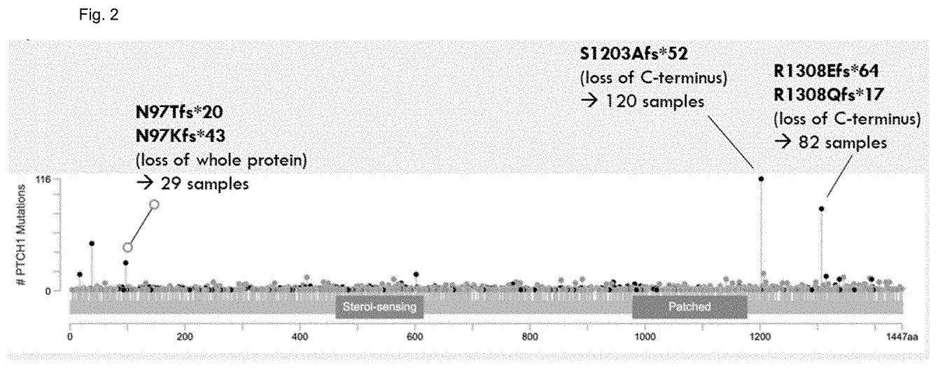

In one aspect, the hedgehog pathway is activated by a loss-of-function mutation in PTCH1, so that PTCH1 fails to inhibit SMO even in the absence of a hedgehog ligand such as sonic hedgehog (SHH). In the absence of a Hh ligand, PTCH1 inhibits SMO, keeping the Hh signaling pathway down-regulated (that is, minimally active or inactive). A loss-of-function mutation causes PTCH1 to fail to inhibit SMO, even in the absence of a Hh ligand. The PTCH1 gene has 23 exons spanning 23 kb and encodes a 1447 amino acid protein with 12 transmembrane domains. The topology and structure of PTCH1 is described in FIG. 1. Loss-of-function mutations are known in the N-terminal cytoplasmic region (residues 1-100), the first transmembrane domain (residues 100-122), and the C-terminal cytoplasmic region (cilia retention) (residues 1176-1447; FIG. 2).

PTHCH1 loss-of-function mutations can be observed in almost all cancer types and are treatable according to the herein disclosed methods. In some embodiments, the cancer with a PTHCH1 loss-of-function mutation is endometrial cancer, colorectal cancer, prostate cancer, melanoma, non-melanoma skin cancer (including basal cell carcinoma), non-small cell lung cancer, esophagogastric cancer, embryonal tumor, glioma, breast cancer, bladder cancer, head and neck cancer, ovarian cancer or mesothelioma.

Table 2 shows the frequency of PTCH1 mutations in these cancers as reported in the American Association for Cancer Research's Project GENIEv9.0 dataset.

| | | | | | | | % LoF |
|---|---|---|---|---|---|---|---|
| | | Total | | | Total | | PTCH1 in |
| | Total | PTCH1 | | Splice | LoF | % LoF | total |
| Cancer type | samples | muts | Truncations | variants | mutants | mutants | indication |
| Colorectal | 11020 | 335 | 109 | 6 | 115 | 34 | 1 |
| Endometrial | 3405 | 191 | 56 | 5 | 61 | 32 | 1.8 |
| NM skin cancer | 12730 | 110 | 42 | 12 | 54 | 49 | 0.42 |
| NSCLC | 971 | 291 | 22 | 15 | 37 | 13 | 4 |
| Esophagogastric | 3075 | 83 | 23 | 1 | 24 | 29 | 0.8 |
| Embryonal | 374 | 39 | 18 | 3 | 21 | 54 | 6 |
| tumor | | | | | | | |
| Glioma | 6750 | 154 | 12 | 9 | 21 | 14 | 0.3 |
| Prostate | 4012 | 71 | 13 | 4 | 17 | 24 | 0.4 |
| Melanoma | 4504 | 153 | 10 | 3 | 13 | 8.5 | 0.3 |
| Breast | 12730 | 143 | 10 | 2 | 12 | 8.4 | 0.1 |
| Bladder | 2719 | 69 | 8 | 1 | 9 | 13 | 0.3 |
| Head and neck | 1681 | 39 | 7 | 2 | 9 | 23 | 0.5 |
| Ovarian | 4230 | 65 | 6 | 3 | 9 | 14 | 0.2 |
| Mesothelioma | 716 | 13 | 4 | 1 | 5 | 39 | 0.7 |

In further embodiments, the cancer with a PTHCH1 loss-of-function mutation is medulloblastoma, pineal cancer, Wilms cancer, bone cancer, cervical cancer, sarcoma, pancreatic cancer, renal cell carcinoma, or small cell lung cancer. For most of these cancer types, treatment with a SMO inhibitor would be futile in more than 90% of patients. Thus, limiting treatment with a SMO inhibitor to patients whose tumor is characterized by an appropriate hedgehog pathway mutation will greatly increase the overall effectiveness of treatment, and allow those patients for whom such treatment would be futile to pursue other more promising treatment without first having to fail treatment with a SMO inhibitor. Even for BCC, where only about 4% of the patients would be excluded from treatment, the avoidance of futile treatment for that 4% is a meaningful benefit.

In some embodiments, the loss-of-function mutation in PTCH1 is a frameshift mutation resulting in a truncated protein. In some embodiments the truncating frameshift mutation comprises N97Tfs*20, N97Kfs*43, S1203Afs*52, R1308Efs*64, R1308Qfs*17, or Y1316Tfs*56. (The notation N97Tfs*20 indicates that the asparagine codon encoding the amino acid at position 97 of the protein has been converted by the frameshift mutation to a threonine codon and that 20 further amino acids are encoded by out of frame codons at which point a termination codon is encountered leading to truncation of the protein. The notation for the other mutants in similarly interpreted. Additional missense, insertion, or deletion mutations that may result in loss-of-function in PTCH1 are listed in FIG. 1C.

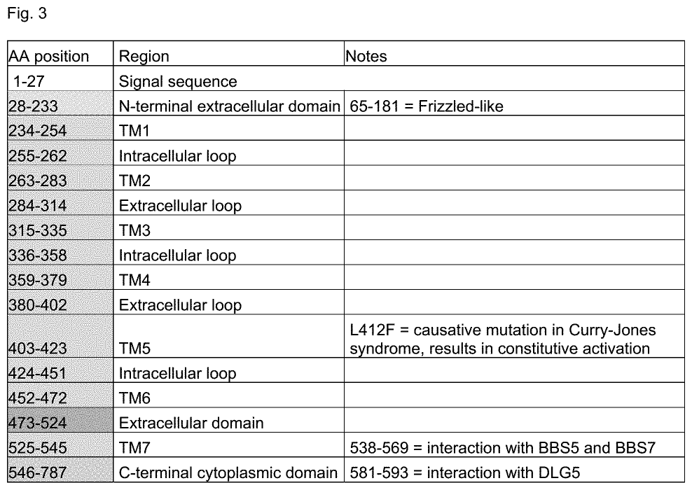

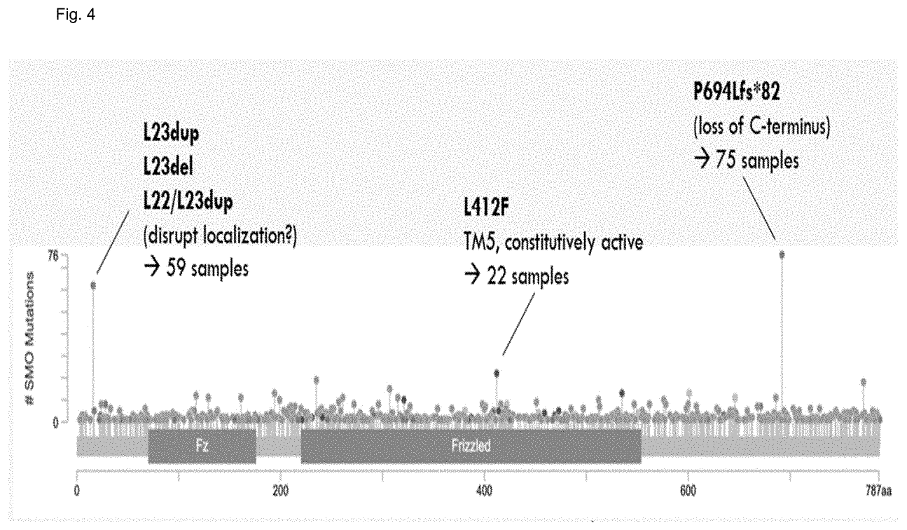

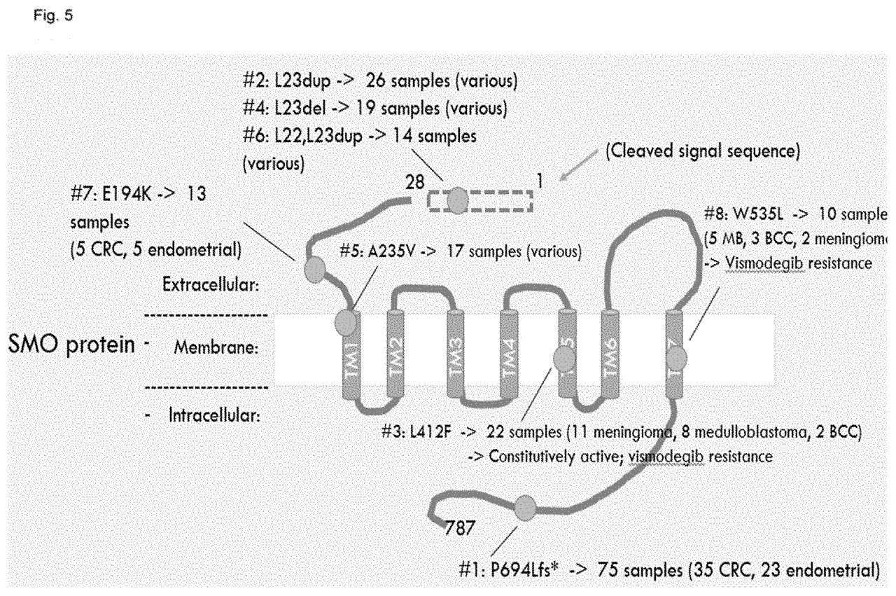

In one aspect, the hedgehog pathway is activated by a gain-of-function mutation in SMO, so that SMO is active even in the presence of PTCH1. Normally, SMO is active only in the absence of inhibition by PTCH1, operating to activate the downstream components of the Hh signaling pathway, such as the Gli family of transcriptional activators. A SMO gain-of-function mutation makes SMO activity independent of regulation by Ptch1. SMO is a 7-transmembrane domain protein of 787 amino acids. It has a topology reminiscent of that of G-protein coupled receptors. The N-terminal region forms the primary interface with PTCH1. FIG. 3 describes the topology of the protein.

SMO gain-of-function mutations can be observed in almost all cancer types and are treatable according to the herein disclosed methods. In some embodiments, the cancer is meningioma, medulloblastoma, basal cell carcinoma, colorectal cancer, or endometrial cancer. In further embodiments, the cancer with a SMO gain-of-function mutation is pineal cancer, head and neck cancer, bladder cancer, glioma, non-small cell lung cancer, Wilms cancer, small cell lung cancer, bone cancer, cervical cancer, mesothelioma, sarcoma, prostate cancer, ovarian cancer, breast cancer, pancreatic cancer, or renal cell carcinoma. For most of these cancer types, treatment with a SMO inhibitor would be futile in the great majority of patients. Thus limiting treatment with a SMO inhibitor to patients whose tumor is characterized by an appropriate hedgehog pathway mutations will greatly increase the overall effectiveness of treatment, and allow those patients for whom such treatment would be futile to pursue other more promising treatment without first having to fail treatment with a SMO inhibitor.

In some embodiments, SMO gain-of-function mutation is an insertion or deletion in the signal sequence. In some embodiments, the insertion or deletion comprises L23dup, L23del, or L22L23dup (that is, an insertion creating a duplication of the leucine residue at position 23, a deletion of the leucine residue at position 23, or an insertion creating a duplication of the leucine residues at position 22 and 23 of the protein, respectively). In some embodiments, the SMO gain-of-function mutation is a missense mutation in the N-terminal extracellular domain. In some embodiments, the missense mutation is E194K (that is, the codon encoding the glutamic acid at residue 94 of the protein has been mutated to instead encode a lysine residue. Analogous notation for other missense mutations is interpreted similarly). In some embodiments, the SMO gain-of-function mutation is a missense mutation in the first transmembrane domain. In some embodiments, the missense mutation in the first transmembrane domain is A235V. In some embodiments, the SMO gain-of-function mutation is a missense mutation in the fifth transmembrane domain. In some embodiments, the missense mutation in the fifth transmembrane domain is L412F. In some embodiments, the SMO gain-of-function mutation is a missense mutation in the seventh transmembrane domain. In some embodiments, the missense mutation in the seventh transmembrane domain is W535L. In some embodiments, the SMO gain-of-function mutation is a frameshift mutation in the C-terminal intracellular domain. In some embodiments, the frameshift mutation in the C-terminal intracellular domain is P694Lfs*. In some embodiments, the SMO gain-of-function mutation is a missense mutation in the C-terminal intracellular domain. In some embodiments, the missense mutation in the C-terminal intracellular domain is R562Q.

In one aspect the hedgehog pathway is activated by a gain-of-function mutation in SHH, so that SHH binds directly to SMO and activates it even in the presence of PTCH1. In particular, many SHH gain-of-function mutations fail to undergo autoprocessing resulting in the persistence of the SHH pro-peptide. This unprocessed precursor form of SHH is sufficient to activate the Hh pathway.

In some embodiments, the herein disclosed mutations constitute means for activating a hedgehog pathway. Some embodiments specifically exclude one or more of these mutations or classes of mutations. For example, in various embodiments, the means for activating a hedgehog pathway do not comprise a loss-of-function mutation in PTCH1, a gain-of-function mutation in SMO, a gain-of-function mutation in SHH, or some combination thereof.

Further embodiments comprise selection of patients based upon their physiological profile. For example, characteristics such as genome or gene expression can be employed when selecting patients. In embodiments, patients are selected based on their PD-L1 expression levels, because in cases, hedgehog pathway activity induces PD-L1 expression on tumor cells, and hedgehog pathway activity data from patients correlates with tumor immunosuppression and resistance to immune checkpoint inhibitors.

Further, disclosed embodiments can achieve synergistic effects. For example, in embodiments, inhibition of hedgehog pathway activity combined with “blocking” of PD-1, for example with an anti-PD-1 or anti-PD-L1 antibody, can provide a synergistic effect in cancer treatment.

The effectiveness of cancer therapy is typically measured in terms of “response.” The techniques to monitor responses can be similar to the tests used to diagnose cancer such as, but not limited to:
d. A lump or tumor involving some lymph nodes can be felt and measured externally by physical examination.
e. Some internal cancer tumors will show up on an x-ray or CT scan and can be measured with a ruler.
f. Blood tests, including those that measure organ function can be performed.
g. A tumor marker test can be done for certain cancers. In some embodiments, the tumor marker test can comprise detection of a hedgehog activating mutation.

Regardless of the test used, whether blood test, cell count, or tumor marker test, it is repeated at specific intervals so that the results can be compared to earlier tests of the same type.

Response to cancer treatment is defined several ways:
h. Complete response—all of the cancer or tumor disappears; there is no evidence of disease. Expression level of tumor marker (if applicable) may fall within the normal range.
i. Partial response—the cancer has shrunk by a percentage but disease remains. Levels of a tumor marker (if applicable) may have fallen (or increased, based on the tumor marker, as an indication of decreased tumor burden) but evidence of disease remains.
j. Stable disease—the cancer has neither grown nor shrunk; the amount of disease has not changed. A tumor marker (if applicable) has not changed significantly.
k. Disease progression—the cancer has grown; there is more disease now than before treatment. A tumor marker test (if applicable) shows that a tumor marker has risen.

Other measures of the efficacy of cancer treatment include:
l. intervals of overall survival (that is time to death from any cause, measured from diagnosis or from initiation of the treatment being evaluated),
m. cancer-free survival (that is, the length of time after a complete response cancer remains undetectable),
n. duration of response (the length of time from the start of a partial or complete response to recurrent or progressive disease),
o. clinical benefit rate (that is, the proportion of patients achieving complete response, partial response, or stable disease) and
p. progression-free survival (that is, the length of time from initiation of treatment to disease progression or death from any cause).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis.

Aspects of the present specification provide, in part, administering a therapeutically effective amount of a compound or a composition disclosed herein. As used herein, the term “therapeutically effective amount” is synonymous with “therapeutically effective dose” and means at least the minimum dose of a compound or composition disclosed herein necessary to achieve a desired therapeutic effect. In some embodiments, it refers to an amount sufficient to inhibit the growth of the cancer. In other embodiments, it refers to an amount sufficient to halt growth of the cancer, that is, to achieve stable disease. In still other embodiments, it refers to an amount sufficient to diminish the size of tumors, that is, to achieve a partial or complete response. In further embodiments, an effective amount is one that prolongs progression-free survival or disease-free survival. In some embodiments, it includes a dose sufficient to reduce a secondary symptom associated with the cancer. An effective dosage or amount of a compound or a composition disclosed herein can readily be determined by the person of ordinary skill in the art considering all criteria (for example, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf. Exemplary dosages are also disclosed herein above.

### EXAMPLES

The following non-limiting examples are provided for illustrative purposes only in order to facilitate a more complete understanding of representative embodiments now contemplated. These examples should not be construed to limit any of the embodiments described in the present specification.

### Example 1

### Clinical Benefit of Taladegib in Advanced Cancer

Approximately 192 study participants have received at least one dose of taladegib in sponsor-initiated and -controlled trials for the treatment of advanced cancers. The clinical trials were:
q. HHBB: Phase 1 dose-escalation study to evaluate the safety and tolerability of taladegib in patients with advanced cancer.
r. HHBE: Phase Ib/2 double-blind randomized study of the Human smoothened (hSmo) antagonist taladegib in combination with carboplatin and etoposide followed by taladegib versus carboplatin and etoposide plus placebo followed by placebo in patients with extensive stage small cell lung cancer (SCLC).
s. HHBH: Phase 1 study of taladegib in Japanese patients with advanced solid tumors.
t. HIPROC: An investigator-initiated Phase 1a/1b dose-escalation study of taladegib in combination with weekly intravenous paclitaxel in patients with advanced solid cancers.
u. HHBF: A Phase 1 ¹⁴C metabolism study of 100 mg of taladegib in healthy subjects.
v. HHBG: A Phase 1 single ascending dose study of taladegib in healthy subjects.

Participants include approximately 156 advanced cancer patients from studies HHBB (N=95), HHBE (N=26), HHBH (N=19), HIPROC (N=16) and 36 healthy volunteers from studies HHBG (N=30) and HHBF (N=6).

These trials (conducted in healthy volunteers, advanced cancer patients as a monotherapy, and advanced cancer patients in combination with anti-neoplastic agents) have demonstrated a manageable safety profile in advanced cancer patients. As a monotherapy, the most commonly observed AEs were nausea, diarrhea, dysgeusia, fatigue, decreased appetite, alopecia, vomiting, muscle spasm, constipation, weight decrease, and headache. As expected, AEs associated with chemotherapy were also observed when taladegib was used in combination with anti-neoplastic agents. On the basis of preclinical and clinical data and information available for molecules in the same therapeutic class, the important potential risks associated with taladegib treatment are considered to be hepatic injury, embryotoxicity and teratogenicity, and rhabdomyolysis.

Across all four multiple-dose studies conducted in patients (HHBB and HHBB addendum, HHBH, HHBE, HIPROC), clinical (RECIST v1.1) responses were observed at all doses. The primary tumor type enrolled was BCC. Additionally, responses in medulloblastoma (monotherapy), SCLC (in combination with carboplatin and etoposide) and ovarian cancer (in combination with paclitaxel) were also observed. A summary of clinical benefit by study is provided in Table 3.

TABLE 3
Clinical Benefit Across All Completed Studies
of Taladegib in Advanced Cancer

| | | | | | Clinical |
|---|---|---|---|---|---|
| Study | N | CR | PR | SD | Benefit |
| HHBB | 84 | 5 (6%) | 17 (20%) | 26 (31%) | 48 (57%) |
| HHBB addendum | 11 | 1 (9%) | 0 | 3 (27%) | 4 (36%) |
| HHBH | 19 | 0 (0%) | 1 (5%) | 4 (21%) | 5 (26%) |
| HHBE | 26 | 0 (0%) | 14 (54%) | 7 (27%) | 21 (81%) |
| HIPROC | 16 | 0 (0%) | 4 (25%) | 6 (38%) | 10 (62%) |
| Total | 156 | 6 (4%) | 42 (27%) | 46 (29%) | 94 (60%) |

R = complete response;
PR = partial response;
SD = stable disease

### Example 2

### A Phase 2, Multi-Center Study Evaluating the Safety and Efficacy of Taladegib in Patients with Advanced Solid Tumors Harboring PTCH1 Loss of Function Mutations

A Simon 2 stage is used to evaluate the efficacy and safety of taladegib in patients with refractory advanced solid tumors characterized by loss of function (LOF) mutations in the PTCH1 gene. Patients with PTCH1 LOF mutations will be identified via genomic sequencing as routinely performed at each participating site. The specific mutation as well as the mutation assay used to detect the mutation will be recorded in the eCRF. Stage 1 (Phase 2a) of this protocol will enroll a total of 44 patients randomized between two dose levels. In the presence of acceptable efficacy, stage 2 (Phase 2b) of this protocol will expand enrollment using a single dose level.

Eligible patients will receive continuous oral dosing of taladegib at a dose of either 200 or 300 mg, once daily, during Phase 2a. The drug will be supplied as tablets containing 100 mg of taladegib. For patients experiencing an adverse event, there will be an option to reduce dosage to a minimum of 100 mg daily. Following Phase 2a, an interim data analysis will be performed. Decision rules for proceeding to Phase 2b and the recommended single dosage for Phase 2b will be defined by the number of objective responses by RECIST 1.1 observed in Phase 2a. Safety and pharmacokinetics will also be considered for dose evaluation.

In the absence of evidence for dose dependence, enrollment will expand to Phase 2b if at least 6 objective responses are observed among the 44 patients treated in Phase 2a. If dose dependence is evident, the selected dose will require at least 4 objective responses among the 22 patients in order to expand to Phase 2b. Phase 2b will enroll approximately 60 patients, if dose dependence is not evident, or approximately 82 patients, if dose dependence is evident, at the recommended Phase 2b dose. This powers the study to test for a favorable objective response rate ≥20% against the null hypothesis of objective response rate ≤10%

In Phase 2a and 2b, patients will be treated once daily during 28-day treatment cycles, until the development of progressive disease (as per Investigator assessment), unacceptable toxicity, withdrawal of consent, death, decision by Investigator, or study termination by the Sponsor.

For tumor measurements, tumors will be assessed at screening by appropriate imaging (e.g., CT, MRI, PET, digital photography). Patients enrolled with a history of CNS tumors should have a brain MRI with contrast at baseline. Tumors will be re-measured every 28±3 days after the first dose of drug and at study termination.

Anti-tumor activity will be evaluated according to the endpoints objective response rate, overall survival, duration of response, clinical benefit rate and progression free survival. Pharmacologic activity will be evaluated as changes in mean Gli1 levels in skin. Specifically, mean Gli1 message inhibition will be determined in skin punch biopsies. Safety evaluation will include adverse events and dose-limiting toxicities (non-hematologic adverse events Grade 3 not including alopecia and fatigue). Pharmacokinetic evaluation will include characterization of the steady-state exposure (C_{trough}) of taladegib.

### Example 3

### Treatment of Cancer with Taladegib

A patient with advanced solid tumors characterized by loss of function (LOF) mutations in the PTCH1 gene is treated with orally administered taladegib and a parenterally administered anti-PD-1 antibody.

### Example 4

### Treatment of Cancer with Taladegib

A patient with refractory advanced solid tumors characterized by loss of function (LOF) mutations in the PTCH1 gene is treated with an orally administered taladegib salt and a parenterally administered anti-PD-1 antibody.

### Example 5

### Treatment of Cancer with Taladegib

A patient with advanced solid tumors and elevated PD-L1 expression is treated with orally administered taladegib and a parenterally administered anti-PD-1 antibody.

### Example 6

### Treatment of Cancer with Taladegib

A patient with refractory advanced solid tumors and elevated PD-L1 expression is treated with an orally administered taladegib salt and a parenterally administered anti-PD-1 antibody.

### Example 7

### Treatment of Cancer with a Compound of Formula 1

A patient with advanced solid tumors and elevated PD-L1 expression is treated with an orally administered compound of Formula 1 and a parenterally administered immune checkpoint inhibitor.

### Example 8

### Treatment of Cancer with a Compound of Formula 1

A patient with refractory advanced solid tumors and elevated PD-L1 expression is treated with an orally administered taladegib salt and a parenterally administered anti-PD-1 antibody.

### Example 9

### Treatment of Cancer with Taladegib

A patient with endometrial cancer and elevated PD-L1 expression is treated with orally administered taladegib and a parenterally administered anti-PD-1 antibody.

### Example 10

### Treatment of Cancer with Taladegib

A patient with esophageal cancer and elevated PD-L1 expression is treated with an orally administered taladegib salt and a parenterally administered anti-PD-1 antibody.

### Example 11

### Treatment of Cancer with a Compound of Formula 1

A patient with prostate cancer and elevated PD-L1 expression is treated with an orally administered compound of Formula 1 and a parenterally administered immune checkpoint inhibitor.

### Example 12

### Treatment of Cancer with a Compound of Formula 1

A patient with basal cell carcinoma and elevated PD-L1 expression is treated with an orally administered taladegib salt and a parenterally administered anti-PD-1 antibody.

### Example 13

### Treatment of Cancer with a Compound of Formula 1

A patient with medulloblastoma and elevated PD-L1 expression is treated with an orally administered taladegib salt and a parenterally administered anti-PD-1 antibody.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating cancer in a patient in need thereof, wherein the cancer is characterized in having an activated hedgehog pathway, comprising administering a Smoothened (SMO) inhibitor.

2. The method of claim 2, wherein said SMO inhibitor comprises a compound of Formula I: 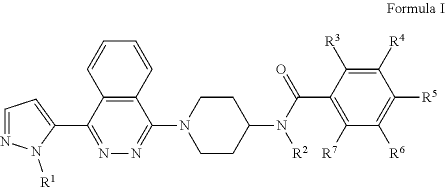
wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

3. The method of claim 1 or 2, further comprising administering an immune checkpoint inhibitor.

4. The method of claim 3, wherein said immune checkpoint inhibitor comprises an anti PD-1 or anti PD-L1 antibody.

5. The method of claim 1, wherein the hedgehog pathway is activated by a loss-of-function mutation in PTCH1.

6. The method of claim 5, wherein the cancer is endometrial cancer, colorectal cancer, esophageal cancer, prostate cancer, basal cell carcinoma, medulloblastoma, hepatocellular carcinoma (HCC), rhabdomyosarcoma, squamous cell carcinomas, pineal cancer, head and neck cancer, bladder cancer, glioma, Wilms cancer, bone cancer, cervical cancer, mesothelioma, sarcoma, ovarian cancer, pancreatic cancer, renal cell carcinoma, non-small cell lung cancer, small cell lung cancer, or breast cancer.

7. The method of claim 5, wherein the loss-of-function mutation in PTCH1 is a frameshift mutation resulting in a truncated protein.

8. The method of claim 7, wherein the truncating frameshift mutation comprises N97Tfs*20, N97Kfs*43, S1203Afs*52, R1308Efs*64, or R1308Qfs*17.

9. The method of claim 5, wherein the loss-of-function mutation in PTCH1 is a missense, insertion, or deletion mutation.

10. The method of claim 9, wherein the missense, insertion, or deletion mutation is L106R, G110R, L128P, L175P, Q177del, H178_A182delinsQ, W197C, T230P, T230I, E237K, G228D, P295S, L297P, E339G, M372R, F367S, N388Y, D390N, A393T, V442E, M449K, triple mutant A472D, G484V, R1319H, L479, S480L, G484R, P504L, G509R, G509D, G509V, G511R, D513Y, D514del, S554R, T557R, P593R, S647C, S647R, K729M, G774R, T778P, Q816del, L822P, 1899V, V908G, W926R, R982Q, T1021C, N10491, A10061_L1062del, T1064M, G1069R, G1069D, V1081M, 91082dup, P1082_V1083del, V1084dup, S1089P, G1093R, H1099R, R1114W, S1132P, S1132Y, A1141V, F1147C, G1167R, P1383L, or E1438D.

11. The method of claim 1, wherein the hedgehog pathway is activated by a gain-of-function mutation in SMO.

12. The method of claim 11, wherein the cancer is endometrial cancer, colorectal cancer, esophageal cancer, prostate cancer, basal cell carcinoma, medulloblastoma, hepatocellular carcinoma (HCC), rhabdomyosarcoma, squamous cell carcinomas, pineal cancer, head and neck cancer, bladder cancer, glioma, Wilms cancer, bone cancer, cervical cancer, mesothelioma, sarcoma, ovarian cancer, pancreatic cancer, renal cell carcinoma, non-small cell lung cancer, small cell lung cancer, or breast cancer.

13. The method of claim 11, wherein the gain-of-function mutation is an insertion or deletion in the signal sequence.

14. The method of claim 13, wherein the insertion or deletion comprises L23dup, L23del, or L22L23dup.

15. The method of claim 11, wherein the gain-of-function mutation is a missense mutation in the N-terminal extracellular domain.

16. The method of claim 15, wherein the missense mutation is E194K.

17. The method of claim 11, wherein the gain-of-function mutation is a missense mutation in the first transmembrane domain.

18. The method of claim 17, wherein the missense mutation is A235V.

19. The method of claim 11, wherein the gain-of-function mutation is a missense mutation in the fifth transmembrane domain.

20. The method of claim 19, wherein the missense mutation is L412F.

21. The method of claim 11, wherein the gain-of-function mutation is a missense mutation in the seventh transmembrane domain.

22. The method of claim 21, wherein the missense mutation is W535L.

23. The method of claim 11, wherein the gain-of-function mutation is a missense mutation in the C-terminal domain.

24. The method of claim 23, wherein the missense mutation is R562Q.

25. The method of claim 11, wherein the gain-of-function mutation is a frameshift mutation in the C-terminal intracellular domain.

26. The method of claim 25, wherein the frameshift mutation is P694Lfs*.

27. The method of any one of claims 1-26, wherein the SMO inhibitor is administered orally.

28. The method of claim 27, wherein the compound of Formula I is taladegib, or a pharmaceutically acceptable salt thereof.

29. The method of claim 28, comprising administering taladegib, or a pharmaceutically acceptable salt thereof, at a dosage of 50-200 mg of the free base of taladegib.

30. A method of treating cancer in a patient in need thereof, wherein the cancer is characterized in having an activated hedgehog pathway, comprising;
administering an effective amount of an I/O antibody; and
administering an effective amount of a compound of Formula I 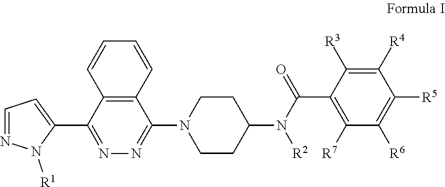
wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

31. The method of claim 30, wherein the hedgehog pathway is activated by a loss-of-function mutation in PTCH1.

32. The method of claim 31, wherein the cancer is endometrial cancer, colorectal cancer, esophageal cancer, prostate cancer, basal cell carcinoma, medulloblastoma, hepatocellular carcinoma (HCC), rhabdomyosarcoma, squamous cell carcinomas, pineal cancer, head and neck cancer, bladder cancer, glioma, Wilms cancer, bone cancer, cervical cancer, mesothelioma, sarcoma, ovarian cancer, pancreatic cancer, renal cell carcinoma, non-small cell lung cancer, small cell lung cancer, or breast cancer.

33. The method of claim 31, wherein the loss-of-function mutation in PTCH1 is a frameshift mutation resulting in a truncated protein.

34. The method of claim 33, wherein the truncating frameshift mutation comprises N97Tfs*20, N97Kfs*43, S1203Afs*52, R1308Efs*64, or R1308Qfs*17.

35. The method of claim 31, wherein the loss-of-function mutation in PTCH1 is a missense, nonsense, insertion, or deletion mutation.

36. The method of claim 35, wherein the missense, insertion, or deletion mutation is L106R, G110R, L128P, L175P, Q177del, H178_A182delinsQ, W197C, T230P, T230I, E237K, G228D, P295S, L297P, E339G, M372R, F367S, N388Y, D390N, A393T, V442E, M449K, triple mutant A472D, G484V, R1319H, L479, S480L, G484R, P504L, G509R, G509D, G509V, G511R, D513Y, D514del, S554R, T557R, P593R, S647C, S647R, K729M, G774R, T778P, Q816del, L822P, 1899V, V908G, W926R, R982Q, T1021C, N10491, A10061_L1062del, T1064M, G1069R, G1069D, V1081M, 91082dup, P1082_V1083del, V1084dup, S1089P, G1093R, H1099R, R1114W, S1132P, S1132Y, A1141V, F1147C, G1167R, P1383L, or E1438D.

37. The method of claim 30, wherein the hedgehog pathway is activated by a gain-of-function mutation in SMO.

38. The method of claim 37, wherein the cancer is endometrial cancer, colorectal cancer, esophageal cancer, prostate cancer, basal cell carcinoma, medulloblastoma, hepatocellular carcinoma (HCC), rhabdomyosarcoma, squamous cell carcinomas, pineal cancer, head and neck cancer, bladder cancer, glioma, Wilms cancer, bone cancer, cervical cancer, mesothelioma, sarcoma, ovarian cancer, pancreatic cancer, renal cell carcinoma, non-small cell lung cancer, small cell lung cancer, or breast cancer.

39. The method of claim 30, wherein the I/O antibody comprises an anti-PD-1 antibody.

40. The method of claim 30, wherein the compound of Formula I is taladegib, or a pharmaceutically acceptable salt thereof.

41. The method of claim 40, comprising administering taladegib, or a pharmaceutically acceptable salt thereof, at a dosage of 50-200 mg of the free base of taladegib.

42. A method of treating cancer in a patient in need thereof, wherein the cancer is characterized in having an activated hedgehog pathway, comprising administering an effective amount of a compound of Formula I 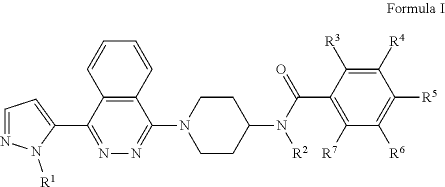
wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

43. A method of treating cancer comprising:
1) identifying a patient having a tumor characterized by comprising means for activating a hedgehog pathway; and
2) administering to the patient an effective amount of a compound of Formula I 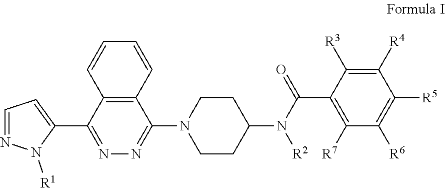
wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

44. A method of treating cancer comprising:
1) detecting means for activating a hedgehog pathway in a patient's tumor; and
2) administering to the patient an effective amount of a compound of Formula I 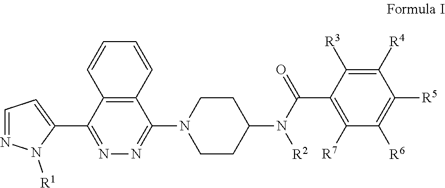
wherein, R¹ is hydrogen or methyl; R² is hydrogen or methyl; R³, R⁴, R⁵, R⁶, or R⁷ are independently hydrogen, fluoro, chloro, cyano, trifluoromethyl, trifluoromethoxy, difluoromethoxy, methylsulfonyl, or trifluoromethylsulfonyl, provided that at least three of R³, R⁴, R⁵, R⁶, and R⁷ are hydrogen; or a pharmaceutically acceptable salt thereof.

45. The method of any one of claims 1-44, further comprising administering an additional anti-cancer agent.

46. The method of any one of claims 1-45, further comprising selecting the patient based on elevated expression of PD-L1.
